(19) United States
(12) Patent Application Publication
(10) Pub. No.: US 2005/0208656 A1
(43) Pub. Date: Sep. 22, 2005

(54) Pattering substrate and cell culture substrate

(76) Inventors: Hideyuki Miyake, Tokyo (JP); Hideshi Hattori, Tokyo (JP); Hironori Kobayashi, Tokyo (JP)

Correspondence Address: SEYFARTH SHAW, 55 EAST MONROE STREET, SUITE 4200, CHICAGO, IL, 60603-5803, US

(21) Appl. No.: 11/001,203

(22) Filed: Nov. 30, 2004

(30) Foreign Application Priority Data
Dec. 5, 2003 (JP) 2003-406788

(51) Int. Cl. C12N 5/02

(52) U.S. Cl. 435/404

(57) ABSTRACT

The present invention intends primarily to provide such as a cell culture patterning substrate that is used to adhere a cell in a highly precise pattern on a base material to culture and a cell culture substrate on which the cell is adhered in a high precision pattern. In order to achieve the above-mentioned object, the present invention provides a patterning substrate having a base material and a cell culture patterning layer that is formed on the base material and at least contains a photocatalyst and a cell adhesion-inhibiting material that has cell adhesion-inhibiting properties that inhibit the cell from adhering and is decomposed or modified owing to an action of the photocatalyst in combination with energy irradiation.

### BACKGROUND OF THE INVENTION

1. Field of the Invention

The present invention relates to a cell culture patterning substrate capable of adhering a cell in a highly precise pattern, a patterning substrate used for forming the cell culture patterning substrate, a coating liquid for patterning substrate used for forming the patterning substrate, and a cell culture substrate on which the cell is adhered in a highly precise pattern.

2. Description of the Related Art

At present, cell cultures of various animals and plants are performed, and also new cell culture methods are in development. The technologies of the cell culture are utilized, such as to elucidate the biochemical phenomena and natures of cells and to produce useful substances. Furthermore, with cultured cells, an attempt to investigate the physiological activity and toxicity of artificially synthesized medicals is under way.

Some cells, particularly a lot of animal cells have the adhesion dependency of adhering to some materials and growing thereon, and cannot survive for a long period under a flotation condition out of organisms. For culturing cells having such adhesion dependency, a carrier to which cells can adhere is necessary, and in general, a plastic culturing plate with uniformly applied cell adhesive proteins such as collagen, fibronectin and the like is used. It is known that these cell adhesive proteins act on cultured cells, make the cells adhere easily, and exert an influence on the form of cells.

On the other hand, there is a technology reported of adhering cultured cells only onto a small part on a base material and arranging them. By such a technology, it is made possible to apply cultured cells to artificial organs, biosensors, bioreactors and the like. As the method of arranging cultured cells, there is a method adopted in which a base material having a surface that forms a pattern different in easiness of adhesion to cells is used, cells are cultured on the surface of this base material and allowed to adhere only onto surfaces processed so that cells adhere, and thereby the cells are arranged.

For example, in Japanese Patent Application Laid-Open (JP-A) No. 2-245181, an electric charge-retaining medium on which an electrostatic pattern is formed is applied to culture cells for the purpose of proliferating nerve cells in a form of circuit, and the like. Furthermore, JP-A No. 3-7576 tries to arrange cultured cells on a surface on which a cell adhesion-inhibiting or cell adhesive photosensitive hydrophilic polymer has been patterned by a photolithography method.

Furthermore, JP-A No. 5-176753 discloses a cell culture base material on which a substance such as collagen and the like affecting on the adhesion ratio and form of cells is patterned, and a method of producing this base material by a photolithography method. By culturing cells on such a base material, a larger amount of cells can be adhered on a surface on which collagen or the like is patterned, to realize patterning of cells.

However, such patterning of cell culture regions may be required to be highly precise depending on applications. In the case of conducting patterning by such as a photolithography method using a photosensitive material as described above, a highly precise pattern can be obtained; however, a cell adhesive material is required to have photosensitivity, and it is difficult in many cases to conduct chemical modification to impart such photosensitivity to, for instance, biopolymers and the like; accordingly, there is a problem in that a range of selectivity of cell adhesive materials is extremely narrowed. Furthermore, in a photolithography method using a photo resist, it is necessary to use a liquid developer and the like, and these affect adversely in culturing cells in some cases.

Furthermore, as a method of forming a highly precise pattern of a cell adhesive material, a Micro Contact Printing method is proposed by George M. Whitesides, Harvard University (for example, U.S. Pat. Nos. 5,512,131 and 5,900,160 and JP-A Nos. 9-240125 and 10-12545). However, there is a problem in that it is difficult to industrially produce a cell culture base material having a pattern of a cell adhesive material using this method.

### SUMMARY OF THE INVENTION

In this connection, it is desired to provide a cell culture patterning substrate used to adhere and culture cells in a highly precise pattern on a base material, a cell culture substrate to which cells are adhered in a highly precise pattern, and the like.

The present invention provides a patterning substrate having a base material; and a cell culture patterning layer that is formed on the base material and contains at least a photocatalyst and a cell adhesion-inhibiting material that has cell adhesion-inhibiting properties that inhibit a cell from adhering and can be decomposed or modified owing to an action of the photocatalyst in combination with energy irradiation.

According to the present invention, since the cell culture patterning layer contains the photocatalyst and the cell adhesion-inhibiting material, by irradiating energy on the cell culture patterning layer, the cell adhesion-inhibiting material can be decomposed or modified, and thereby a region where an adhesiveness with a cell is excellent can be formed. Therefore, according to the invention, without invoking a special device or a complicated process, a patterning substrate on which a region where the cell adhesion-inhibiting material is decomposed or modified and the adhesiveness with a cell is excellent and a region that contains the cell adhesion-inhibiting material and is low in the adhesiveness with a cell can be readily formed can be formed.

In the above-mentioned invention, on the base material, a light-shielding portion can be formed in pattern. In this case, when energy is irradiated from a base material side, energy can be irradiated only on a cell culture patterning layer in a region where the light-shielding portion is not formed; accordingly, a patterning substrate on which a pattern where the cell adhesion-inhibiting material remains in a pattern according to which the light-shielding portion is formed can be formed can be obtained.

In the present invention, the cell culture patterning layer preferably contains a binder. Thereby, the characteristics such as imparting the strength to the cell culture patterning layer and controlling the adhesiveness with a cell in a region where energy is irradiated can be imparted.

In the present invention, the cell culture patterning layer, at least after energy is irradiated, preferably contains a cell adhesive material having the adhesiveness with a cell. Thereby, by irradiating energy, the adhesiveness with a cell in a region where the cell adhesion-inhibiting material of the cell culture patterning layer is decomposed or modified can be made higher. Thereby, only in a region where energy is irradiated, the cell can be adhered in high precision.

The present invention provides a cell culture patterning substrate having a base material and a cell culture pattern layer formed on the base material, wherein the cell culture pattern layer having a cell adhesion-inhibiting portion that contains at least a photocatalyst and a cell adhesion-inhibiting material that has cell adhesion-inhibiting properties that inhibit a cell from adhering and can be decomposed or modified owing to an action of the photocatalyst in combination with energy irradiation; and a cell adhesion portion that contains at least the photocatalyst and where the cell adhesion-inhibiting material is decomposed or modified.

According to the present invention, since the cell adhesion-inhibiting portion and the cell adhesion portion are formed, without invoking a special device or a processing liquid and the like that adversely affects on the cell, a cell culture patterning substrate capable of readily adhering the cell only on the cell adhesion portion can be formed.

In the present invention, on the base material, a light-shielding portion may be formed in a pattern same as to the cell adhesion-inhibiting portion. Thereby, the cell adhesion portion in the cell culture pattern layer, for instance, by irradiating energy from a base material side over an entire surface, can be readily formed.

In the present invention, the cell culture pattern layer preferably contains a binder. Thereby, characteristics such as that the strength of the cell culture pattern layer can be improved and an adhesiveness with a cell on the cell adhesion portion can be controlled can be imparted.

In the present invention, the cell culture pattern layer, at least after energy is irradiated, preferably contains a cell adhesive material having the adhesiveness with a cell. Thereby, the adhesiveness with a cell on the cell adhesion portion can be made higher.

The present invention provides a cell culture substrate wherein the cell is adhered onto the cell adhesion portion in any of the above-mentioned cell culture patterning substrates.

According to the present invention, the cell does not adhere onto the cell adhesion-inhibiting portion of the cell culture patterning substrate and adhere only onto the cell adhesion portion; accordingly, without applying a complicated process or using a processing liquid and the like that may adversely affect on the cell, the cell culture substrate on which the cell are adhered in a highly precise pattern can be readily prepared.

The present invention provides a coating liquid for patterning substrate that contains at least a photocatalyst and a cell adhesion-inhibiting material that has cell adhesion-inhibiting properties that inhibit a cell from adhering and can be decomposed or modified owing to an action of the photocatalyst in combination with energy irradiation.

According to the present invention, owing to the inclusion of the photocatalyst and the cell adhesion-inhibiting material, for instance, when the coating liquid for patterning substrate is coated on the base material followed by such as irradiating energy in pattern, owing to an action of the photocatalyst in combination with energy irradiation, the cell adhesion-inhibiting material is decomposed or modified, and thereby a region where the adhesiveness with a cell is excellent and a region where the adhesiveness with a cell is low owing to the cell adhesion-inhibiting properties of the cell adhesion-inhibiting material can be readily formed.

In the present invention, the coating liquid for patterning substrate preferably contains a binder. Thereby, when the coating liquid for patterning substrate is coated to form a layer, for instance, a layer can be readily formed and the strength of the layer can be improved.

In the present invention, the coating liquid for patterning substrate, at least after energy is irradiated, preferably contains a cell adhesive material that has an adhesiveness with a cell. Thereby, when the coating liquid for patterning substrate is coated to form a layer, the adhesiveness with a cell in a region where energy is irradiated and thereby the cell adhesion-inhibiting material is decomposed or modified can be made higher; accordingly, only in the region where energy is irradiated cells can be adhered in a highly precise pattern.

### BRIEF DESCRIPTION OF THE DRAWINGS

FIG. 1 is a schematic sectional view showing one example of the patterning substrate according to the present invention.

FIG. 2 is a schematic sectional view showing another example of the patterning substrate according to the invention.

FIG. 3 is a schematic sectional view showing one example of the cell culture patterning substrate according to the invention.

FIG. 4 is a schematic sectional view showing another example of the cell culture patterning substrate according to the invention.

FIGS. 5A to 5C are process charts showing one example of a method of manufacturing the cell culture pattern layer in the cell culture patterning substrate according to the invention.

FIG. 6 is a schematic sectional view showing one example of the cell culture substrate according to the invention.

### DESCRIPTION OF THE PREFERRED EMBODIMENTS

The present invention relates to a cell culture patterning substrate on which a cell can be adhered in highly precise pattern, a patterning substrate that is used to form the cell culture patterning substrate, a coating liquid for patterning substrate that is used to form the patterning substrate, a cell culture substrate on which the cell are adhered in highly precise pattern, and the like. Hereinafter, these each will be explained separately.

A. Coating Liquid for Patterning Substrate

First, a coating liquid for patterning substrate will be explained. The coating liquid for patterning substrate according to the present invention contains at least a photocatalyst and a cell adhesion-inhibiting material that has cell adhesion-inhibiting properties that inhibit a cell from adhering and can be decomposed or modified owing to an action of the photocatalyst in combination with energy irradiation.

In the coating liquid for patterning substrate according to the present invention, the cell adhesion-inhibiting material having the cell adhesion-inhibiting properties that inhibit the cell from adhering is contained; accordingly, for instance, when the coating liquid for patterning substrate is applied on a base material to form a layer, an adhesiveness with a cell can be made low. Furthermore, the cell adhesion-inhibiting material, since it can be decomposed or modified owing to an action of the photocatalyst in combination with energy irradiation, when energy is irradiated on a layer formed on the base material, a region where energy is irradiated can be made one where the cell adhesion-inhibiting material is not contained or less contained. Accordingly, according to the invention, when the coating liquid for patterning substrate according to the invention is coated to form a layer followed by such as irradiating energy in a pattern, a region where the adhesiveness with a cell is low and a region where the adhesiveness with a cell is excellent can be formed.

Hereinafter, respective materials used in the coating liquid for patterning substrate according to the present invention will be explained.

1. Photocatalyst

At the beginning, a photocatalyst used in a coating liquid for patterning substrate according to the present invention will be explained. The photocatalyst used in the invention, as far as it can decompose or modify a cell adhesion-inhibiting material described below owing to an action of the photocatalyst in combination with energy irradiation, is not particularly restricted.

An action mechanism of the photocatalyst typical in titanium oxide described below is not necessarily clear. However, it can be considered that a carrier generated by irradiation of light directly reacts with a nearby compound or, owing to an active oxygen species generated under the presence of oxygen, water, a chemical structure of an organic material is caused to change. In the invention, the carrier is considered to affect on the cell adhesion-inhibiting material described below.

As the photocatalyst that can be used in the present invention, specifically, for instance, titanium dioxide (TiO₂), zinc oxide (ZnO), tin oxide (SnO₂), strontium titanate (SrTiO₃), tungsten oxide (WO₃), bismuth oxide (Bi₂O₃) and iron oxide (Fe₂O₃) that are known as photo-semiconductors can be cited. These can be used singularly or in combination of at least two kinds.

In the present invention, in particular, titanium dioxide, owing to a large band gap, chemical stability, non-toxicity, and easy availability, can be preferably used. There are two types of titanium dioxide, anatase type and rutile type, and both can be used in the invention; however, the anatase type titanium dioxide is more preferable. An excitation wavelength of the anatase type titanium dioxide is 380 nm or less.

As such anatase type titanium dioxide, for instance, an anatase titania sol of hydrochloric acid deflocculation type (trade name: STS-02, manufactured by Ishihara Sangyo Kaisha, Ltd., average particle diameter: 7 nm, and trade name: ST-KO1, manufactured by Ishihara SangyoKaisha, Ltd.), an anatase titania sol of nitric acid deflocculation type (trade name: TA-15, manufactured by Nissan Chemical Industries Ltd., average particle diameter: 12 nm) and the like can be cited.

The smaller is a particle diameter of the photocatalyst, the better, because a photocatalyst reaction is caused more effectively. An average particle diameter of the photocatalyst is preferably 50 nm or less, and one having an average particle diameter of 20 nm or less can be particularly preferably used.

A content of the photocatalyst in the coating liquid for patterning substrate according to the present invention can be set in the range of 5 to 95% by weight, preferably of 10 to 60% by weight, and more preferably of 20 to 40% by weight. Thereby, when the coating liquid for patterning substrate is coated and a layer is formed, the cell adhesion-inhibiting material in a region where energy is irradiated can be decomposed or modified.

The photocatalyst used in the present invention is preferably excellent in an adhesiveness with a cell. Thereby, a region where the photocatalyst is exposed owing to the decomposition and the like of the cell adhesion-inhibiting material described below can be used as a region where the adhesiveness with a cell is excellent.

2. Cell Adhesion-Inhibiting Material

A cell adhesion-inhibiting material that is used in the coating liquid for patterning substrate according to the present invention will be explained. The cell adhesion-inhibiting material used in the coating liquid for patterning substrate according to the invention, as far as it has cell adhesion-inhibiting properties that inhibit a cell from adhering and can be decomposed or modified owing to an action of a photocatalyst in combination with energy irradiation, is not particularly restricted in the kind and the like.

Here, “having the cell adhesion-inhibiting properties” means having a property that inhibits the cell from adhering to the cell adhesion-inhibiting material, and, when an adhesiveness with a cell is different depending on the kind of cells and the like, means to have the property of inhibiting from adhering with a target cell.

As the cell adhesion-inhibiting material used in the present invention, one that has such cell adhesion-inhibiting properties and can be decomposed or modified owing to an action of the photocatalyst in combination with energy irradiation to lose the cell adhesion-inhibiting properties or to become excellent in the adhesiveness with a cell can be used.

As the cell adhesion-inhibiting material, materials high in, for instance, the hydration ability can be used. When the material high in the hydration ability is used, water molecules gather in the vicinity of the cell adhesion-inhibiting material to form a hydrated layer. Normally, such material high in the hydration ability is higher in an adhesiveness with water molecules than that with the cell; accordingly, the cells cannot adhere to the material high in the hydration ability, resulting in one low in the adhesiveness with a cell. Here, “the hydration ability” means a property of hydrolyzing with water molecules, and “the hydration ability being high” means that it can readily hydrolyze with water molecules.

As materials that are high in the hydration ability and can be used as the cell adhesion-inhibiting material, for instance, polyethylene glycol, amphoteric ion materials having a betaine structure or the like, and phosphatide-containing materials and the like can be cited. In the case of such a material being used as the cell adhesion-inhibiting material, when energy is irradiated, owing to an action of the photocatalyst, the cell adhesion-inhibiting material is decomposed, modified or the like, the hydrated layer on a surface comes off, and thereby the cell adhesion-inhibiting material is rendered one that does not have the cell adhesion-inhibiting properties.

A surfactant having a water repellent or oil repellent organic group that can be decomposed owing to an action of the photocatalyst also can be used. As such surfactant, hydrocarbons base surfactants such as the respective series of NIKKO L, BL, BC, BO, and BB manufactured by Nikko Chemicals Co., Ltd., and fluorine base or silicone base nonionic surfactants such as ZONYL FSN and FSO manufacture by Du Pont Kabushiki Kaisya, Surflon S-141 and 145 manufactured by ASAHI GLASS CO., LTD., Megaface F-141 and 144 manufactured by DAINIPPON INK AND CHEMICALS, Inc., FTERGENT F-200 and F-251 manufactured by NEOS, UNIDYNE DS-401 and 402 manufactured by DAIKIN INDUSTRIES, Ltd., and Fluorad FC-170 and 176 manufactured by 3M can be cited, and furthermore cationic surfactants, anionic surfactants and amphoteric surfactants also can be used.

In the case of such a material being use as the cell adhesion-inhibiting material, when the coating liquid for patterning substrate is coated to form a layer, the cell adhesion-inhibiting material is distributed unevenly on a surface. Thereby, the water repellency or oil repellency of the surface can be made higher; accordingly, one small in the interaction with a cell and low in the adhesiveness with a cell can be obtained. Furthermore, when energy is irradiated on the layer, owing to an action of the photocatalyst, the cell adhesion-inhibiting material is readily decomposed to expose the photocatalyst, resulting in one that does not have the cell adhesion-inhibiting properties.

In the present invention, as the cell adhesion-inhibiting material, one that can be improved in the adhesiveness with a cell owing to an action of the photocatalyst in combination with energy irradiation is particularly preferably used. As such cell adhesion-inhibiting materials, for instance, materials having the oil repellency or water repellency can be cited.

In the case of, as the cell adhesion-inhibiting material, a material having the water repellency or the oil repellency being used, owing to the water repellency or the oil repellency of the cell adhesion-inhibiting material, one in which an interaction such as a hydrophobic interaction between the cell and the cell adhesion-inhibiting material is small and the adhesiveness with a cell is low can be obtained.

As a material having such water repellency or the oil repellency, for instance, one where a skeleton has such a high bond energy that cannot be decomposed by an action of the photocatalyst and that has a water repellent or oil repellent organic substitution group that can be decomposed owing to an action of the photocatalyst can be cited.

As one that has the skeleton having such high bond energy that cannot be decomposed owing to an action of the photocatalyst and the water repellent or oil repellent organic substitution group that can be decomposed owing to an action of the photocatalyst, for instance, (1) organopolysiloxane that exhibits large strength by hydrolyzing or polycondensating chloro, alkoxysilane or the like owing to a sol-gel reaction and the like, and (2) organopolysiloxane in which reactive silicones are crosslinked can be cited.

In the case of the (1), it is preferable to be organopolysiloxane that is a hydrolysis condensate or cohydrolysis condensate of at least one kind of silicon compounds expressed by a general formula:
YₙSiX₍₄₋ₙ₎
(Here, Y denotes an alkyl group, fluoroalkyl group, vinyl group, amino group, phenyl group or epoxy group, or organic groups including these, and X denotes an alkoxyl group, acetyl group or halogen. n is an integer of 0 to 3.). The number of carbons of the group expressed with Y is preferably in the range of 1 to 20, and the alkoxy group expressed with X is preferably a methoxy group, ethoxy group, propoxy group or butoxy group.

As the organic groups, in particular, polysiloxane containing a fluoroalkyl group can be preferably used. Specifically, a hydrolysis condensate or cohydrolysis condensate of at least one kind of fluoroalkylsilanes below can be cited. Ones generally known as the fluorinated silane coupling agents can be used.
CF₃(CF₂)₃CH₂CH₂Si(OCH₃)₃;
CF₃(CF₂)₅CH₂CH₂Si(OCH₃)₃;
CF₃(CF₂)₇CH₂CH₂Si(OCH₃)₃;
CF₃(CF₂)₉CH₂CH₂Si(OCH₃)₃;
(CF₃)₂CF(CF₂)₄CH₂CH₂Si(OCH₃)₃;
(CF₃)₂CF(CF₂)₆CH₂CH₂Si(OCH₃)₃;
(CF₃)₂CF(CF₂)₈CH₂CH₂Si(OCH₃)₃;
CF₃(C₆H₄)C₂H₄Si(OCH₃)₃;
CF₃(CF₂)₃(C₆H₄)C₂H₄Si(OCH₃)₃;
CF₃(CF₂)₅(C₆H₄)C₂H₄Si(OCH₃)₃;
CF₃(CF₂)₇(C₆H₄)C₂H₄Si(OCH₃)₃;
CF₃(CF₂)₃CH₂CH₂SiCH₃(OCH₃)₂;
CF₃(CF₂)₅CH₂CH₂SiCH₃(OCH₃)₂;
CF₃(CF₂)₇CH₂CH₂SiCH₃(OCH₃)₂;
CF₃(CF₂)₉CH₂CH₂SiCH₃(OCH₃)₂;
(CF₃)₂CF(CF₂)₄CH₂CH₂SiCH₃(OCH₃)₂;
(CF₃)₂CF(CF₂)₆CH₂CH₂SiCH₃(OCH₃)₂;
(CF₃)₂CF(CF₂)₈CH₂CH₂SiCH₃(OCH₃)₂;
CF₃(C₆H₄)C₂H₄SiCH₃(OCH₃)₂;
CF₃(CF₂)₃(C₆H₄)C₂H₄SiCH₃(OCH₃)₂;
CF₃(CF₂)₅(C₆H₄)C₂H₄SiCH₃(OCH₃)₂;
CF₃(CF₂)₇(C₆H₄)C₂H₄SiCH₃(OCH₃)₂;
CF₃(CF₂)₃CH₂CH₂Si(OCH₂CH₃)₃;
CF₃(CF₂)₅CH₂CH₂Si(OCH₂CH₃)₃;
CF₃(CF₂)₇CH₂CH₂Si(OCH₂CH₃)₃;
CF₃(CF₂)₉CH₂CH₂Si(OCH₂CH₃)₃;
CF₃(CF₂)₇SO₂N(C₂H₅)C₂H₄CH₂Si(OCH₃)₃

When polysiloxane containing such a fluoroalkyl group is used as the cell adhesion-inhibiting material, one high in the water repellency or oil repellency can be formed, resulting in rendering one small in the interaction with a cell and low in the adhesiveness with a cell. Furthermore, when energy is irradiated on such a material, readily, fluorine and the like can be removed and an OH group and the like can be introduced on a surface to render the interaction with a cell larger; accordingly, the adhesiveness with a cell can be made excellent.

As the reactive silicone according to the (2), compounds having a skeleton expressed by a general formula below can be cited.

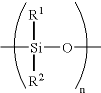

In the above general formula, n denotes an integer of 2 or more, R¹and R² each represents a substituted or nonsubstituted alkyl group, aryl group or cyanoalkyl group having 1 to 10 carbons, and a vinyl, phenyl and halogenated phenyl occupy 40% or less by mole ratio to a total mole. Furthermore, one in which R¹ and R² each is a methyl group is preferable because the surface energy is the lowest, and a methyl group is preferably contained 60% or more by mole ratio. Still furthermore, a chain terminal or side chain has at least one or more reactive group such as a hydroxyl group in a molecular chain.

Together with the organopolysiloxane, a stable organosilicium compound that does not cause a crosslinking reaction such as dimethylpolysiloxane may be blended separately.

When such reactive silicone is used, one high in the water repellency or oil repellency can be obtained; that is, one small in the interaction with a cell and low in the adhesiveness with a cell can be obtained. Furthermore, when energy is irradiated on such a material, readily, a substituent group can be removed and an OH group and the like can be introduced on a surface to render the interaction with a cell larger; accordingly, the adhesiveness with a cell can be made excellent.

Such cell adhesion-inhibiting material is preferably contained in the coating liquid for patterning substrate in the range of 0.01 to 95% by weight and particularly preferably in the range of 1 to 10% by weight. Thereby, a region that contains the cell adhesion-inhibiting material can be made a region low in the adhesiveness with a cell.

When energy is irradiated on the cell adhesion-inhibiting material such as mentioned above to decompose or modify and thereby to use as one excellent in the adhesiveness with a cell, the energy may be irradiated to decompose or modify the cell adhesion-inhibiting material to an extent that makes the adhesiveness with target cell excellent, and there is no need of completely decomposing or modifying the cell adhesion-inhibiting material.

3. Coating Liquid for Patterning Substrate

A coating liquid for patterning substrate according to the present invention will be explained. The coating liquid for patterning substrate according to the invention, as far as it contains the above-mentioned photocatalyst and the cell adhesion-inhibiting material, is not particularly restricted. As needs arise, it may appropriately contain a binder and the like.

In the present invention, in particular in the coating liquid for patterning substrate, a binder or a cell adhesive material that becomes excellent in an adhesiveness with a cell at least after energy is irradiated is preferably contained. When the binder is contained, various characteristics such that when the coating liquid for patterning substrate is coated on, for instance, a base material or the like, the coating can be easily applied and a formed layer can be imparted with the strength and the durability can be realized.

In the case of the coating liquid for patterning substrate according to the present invention containing the cell adhesive material, when it is coated and a layer is formed, the adhesiveness with a cell of a region where energy is irradiated and a cell adhesion-inhibiting material is decomposed or modified can be improved further. Such cell adhesive material may play a role as the binder.

Hereinafter, binders and cell adhesive materials that can be used in the coating liquid for patterning substrate according to the present invention will be explained.

(Binder)

At the beginning, a binder used in the present invention will be explained. The binder used in the invention, as far as it does not inhibit the characteristics of the photocatalyst and the cell adhesion-inhibiting material, is not particularly restricted. Ones in which, for instance, coating properties, the strength and durability when it is formed into a layer, and the like are tailored to characteristics necessary for the coating liquid for patterning substrate can be used. Here, the cell adhesion-inhibiting material may be one that plays a function as the binder.

As such binder, for instance, one in which a main skeleton has such a high bond energy that cannot be decomposed owing to an action of the photocatalyst can be used. Specifically, polysiloxane and the like that does not have an organic substituent or has an organic substituent to an extent that does not affect on the adhesiveness can be cited. These can be obtained by hydrolyzing or polycondensating tetramethoxysilane, tetraethoxysilane and the like.

In the present invention, such binder may be preferably contained in the coating liquid for patterning substrate in the range of 5 to 95% by weight, among the above, 40 to 90% by weight, and in particular in the range of 60 to 80% by weight. Thereby, characteristics such that the coating liquid for patterning substrate can be readily coated, a layer obtained by coating the coating liquid for patterning substrate is imparted with the strength and the like can be exhibited.

(Cell Adhesive Material)

A cell adhesive material used in the present invention, as far as it has the adhesiveness with a cell at least after energy is irradiated, is not particularly restricted. As mentioned above, it may be one that is used as the above-mentioned binder or one that is used separately from the above-mentioned binder. Furthermore, for instance, it may be one that has the excellent adhesiveness with a cell prior to energy irradiation or one that becomes excellently adhesive with a cell owing to an action of the photocatalyst in combination with energy irradiation. Here, “having the adhesiveness with a cell” means to excellently adhere to a cell and, when the adhesiveness with a cell is different depending on kinds and the like of cells, means to excellently adhere to a target cell.

In the present invention, as far as the cell adhesive material has excellent adhesiveness with a cell at least after energy is irradiated, it may be one of which adhesiveness with a cell is made excellent owing to physical interactions such as an hydrophobic interaction, electrostatic interaction, hydrogen bonding, van der Waals force and the like or owing to biological characteristics.

As a material that has the adhesiveness with a cell owing to the physical interactions, specifically, temperature sensitive polymers such as hydrophilic polystyrene and poly (N-isopropylacrylamide), basic polymers such as polylysine, basic compounds such as aminopropyltriethoxysilane, N-(2-aminoethyl)-3-aminopropyltrimethoxysilane and condensates and the like including these can be cited.

As a material having the adhesiveness with a cell owing to the biological characteristics, specifically, fibronectin, laminin, tenascin, vitronectin, RGD (arginine-glycine-asparagine acid) sequence containing peptide, YIGSR (tyrosine-isoleucine-glycine-serine-arginine) sequence containing peptide, collagen, atelocollagen, gelatin and the like can be cited.

In the present invention, such cell adhesive material is contained in the coating liquid for patterning substrate normally in the range of 0.01 to 95% by weight, and preferably in the range of 1 to 10% by weight. Thereby, when the coating liquid for patterning substrate is applied to form a layer, the adhesiveness with a cell in a region where energy is irradiated can be made more excellent. Furthermore, when a material that has, prior to energy irradiation, the excellent adhesiveness with a cell is used as the cell adhesive material, in a region where energy is not irradiated after the coating liquid for patterning substrate is coated to form a layer, the cell adhesive material is preferably contained to an extent that does not damage the cell adhesion-inhibiting properties of the cell adhesion-inhibiting material. The cell adhesion-inhibiting material preferably has the surface activity. In the course of coating and drying the coating liquid for patterning substrate, the cell adhesion-inhibiting material becomes unevenly distributed to present on a surface of coating, resulting in excellent cell adhesion-inhibiting properties.

B. Patterning Substrate

A patterning substrate according to the present invention will be explained. The patterning substrate according to the invention includes a base material; and a cell culture patterning layer that is formed on the base material and contains at least a photocatalyst and a cell adhesion-inhibiting material that has cell adhesion-inhibiting properties that inhibit a cell from adhering and can be decomposed or modified owing to an action of the photocatalyst in combination with energy irradiation.

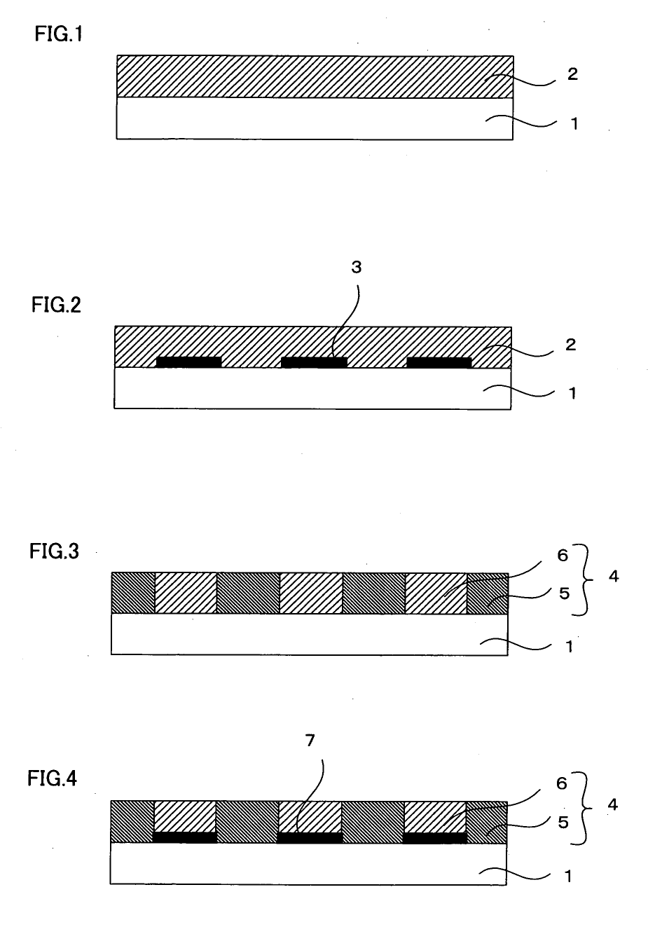

The patterning substrate according to the present invention, as shown in, for instance, FIG. 1, has a base material 1 and a cell culture patterning layer 2 formed on the base material. According to the invention, since the cell culture patterning layer contains the cell adhesion-inhibiting material that has the cell adhesion-inhibiting properties, it can be made one to which the cell does not adhere. Furthermore, when energy is irradiated on the cell culture patterning layer, the cell adhesion-inhibiting material can be decomposed or modified owing to an action of the photocatalyst in combination with energy irradiation and thereby can be rendered one to which the cell adhere. Accordingly, without necessitating a special device or a complicated process, by irradiating energy in a pattern, a region where the cell adhesion-inhibiting material is decomposed and removed to be excellent in an adhesiveness with a cell and a region where the cell adhesion-inhibiting material remains and the adhesiveness with a cell is low can be easily formed.

In the patterning substrate according to the present invention, as shown in, for instance, FIG. 2, a light-shielding portion 3 may be formed on the base material 1. When the light-shielding portion is formed, in the case of energy being irradiated from a base material side, energy is not irradiated on the cell culture patterning layer on a region where the light-shielding portion is formed. Thereby, only the cell adhesion-inhibiting material on the region where the light-shielding portion is not formed can be decomposed or modified; accordingly, without using a photomask and the like, a region where the cell adhere and a region where the cell does not adhere can be readily formed.

Hereinafter, the respective configurations of the patterning substrate according to the present invention will be explained.

1. Cell Culture Patterning Layer

A cell culture patterning layer used in the present invention will be explained. The cell culture patterning layer used in the invention, as far as it is formed on the base material described below and contains at least a photocatalyst and a cell adhesion-inhibiting material that has cell adhesion-inhibiting properties that inhibit a cell from adhering and can be decomposed or modified owing to an action of the photocatalyst in combination with energy irradiation, is not particularly restricted.

The cell culture patterning layer can be formed by coating, for instance, the above-mentioned coating liquid for patterning substrate to form a layer. In particular, the layer is preferably one that is formed with a coating liquid for patterning substrate that contains a cell adhesive material of which an adhesiveness with a cell is excellent at least after energy is irradiated. Thereby, when energy is irradiated on the cell culture patterning layer, the adhesiveness with a cell in a region where energy is irradiated can be made higher, and in a region where energy is irradiated, the cell can be adhered with a highly precise pattern.

The coating liquid for patterning substrate and the like can be applied according to general methods. For instance, a spin coat method, spray coat method, dip coat method, roll coat method, bead coat method and the like can be used.

The photocatalyst, cell adhesion-inhibiting material and binder and the like contained in the cell culture patterning layer used in the present invention are identical to those described in the section of “A. Coating liquid for patterning substrate”. Accordingly, explanations thereof are not repeated.

A film thickness of the cell culture patterning layer, though properly selected depending on the kind and the like of the patterning substrate, is normally in the range of about 0.01 to 1.0 μm, and among these can be made in the range of about 0.1 to 0.3 μm.

2. Base Material

In the next place, a base material used in the present invention will be explained. The base material used in the invention, as far as it is a layer on which the cell culture patterning layer can be formed, is not particularly restricted. For instance, inorganic materials such as metal, glass and silicium, and organic materials and the like typical in plastics can be used.

The flexibility and the like of the base material are properly selected according to the kind of the patterning substrate, applications or the like. Furthermore, the transparency of the base material is properly selected depending on such as the kind of the patterning substrate, or a direction in which energy that is irradiated to decompose or modify the cell adhesion-inhibiting material is irradiated. For instance, when the base material has such as the light-shielding portion and the energy is irradiated from a base material side, the base material has the transparency.

On the base material according to the present invention, as mentioned above, a light-shielding portion may be formed. The light-shielding portion that can be used in the invention, as far as it can shield energy that is irradiated on the patterning substrate, is not particularly restricted. For instance, a metal thin film that is made of chromium or the like and formed into a thickness of about 1000 to 2000 Å by a sputtering method, a vacuum deposition method or the like is formed and patterned to form a shielding portion. As the patterning method, an ordinary patterning method such as the sputtering can be used.

A method may be one by which a layer that contains light-shielding particles such as carbon particulates, metal oxides, inorganic pigments and organic pigments in a resin binder is formed in a pattern. As the resin binders that can be used, a polyimide resin, acrylic resin, epoxy resin, polyacrylamide, polyvinyl alcohol, gelatin, casein, cellulose and the like can be used singularly or in combination of two or more kinds, and furthermore a photosensitive resin and an O/W emulsion type resin composition such as emulsified reactive silicone can be used. A thickness of such resinous light-shielding portion can be set in the range of 0.5 to 10 μm. As a method of patterning such resinous light-shielding portion, methods such as a photolithography method and a printing method that are generally used can be used.

The light-shielding portion may be formed on a surface of a side where the cell culture patterning layer is formed of the base material or may be formed on a surface of an opposite side.

In the case of forming the light-shielding portion, a primer layer may be formed between the cell culture patterning layer and the light-shielding portion. An action and function of the primer layer is not necessarily clear. However, it is considered that it has a function that when the primer layer is formed, impurities from the light-shielding portions and openings present between the light-shielding portions that become a factor that inhibits the decomposition or modification of the cell adhesion-inhibiting material in the cell culture patterning layer owing to an action of the photocatalyst, in particular, impurities such as residues generated when the light-shielding portions are patterned, metal and metal ion are inhibited from diffusing. Accordingly, when the primer layer is formed, the cell adhesion-inhibiting material can be decomposed or modified with high sensitivity, resulting in obtaining a high resolution pattern.

The primer layer in the present invention inhibits the impurities present not only in the light-shielding portions but also in the openings formed between the light-shielding portions from adversely affecting on an action of the photocatalyst; accordingly, the primer layer is preferably formed over an entire surface of the light-shielding portion including the openings.

The primer layer according to the present invention, as far as it is formed so that the light-shielding portion and the cell culture patterning layer may not be brought into contact, is not particularly restricted.

A material that forms the primer layer, though not particularly restricted, is preferably an inorganic material that is not likely to be decomposed owing to an action of the photocatalyst. Specifically, amorphous silica can be cited. When such amorphous silica is used, a precursor of the amorphous silica is preferably a silicon compound that is represented by a general formula, SiX₄, X being halogen, methoxy group, ethoxy group, acetyl group or the like, silanol that is a hydrolysate thereof, or polysiloxane having an average molecular weight of 3000 or less.

A film thickness of the primer layer is preferably in the range of 0.001 to 1 μm and particularly preferably in the range of 0.001 to 0.1 μm.

3. Patterning Substrate

Subsequently, a patterning substrate according to the present invention will be explained. The patterning substrate according to the invention, as far as it is one in which a cell culture patterning layer is formed on the above-mentioned base material, is not particularly restricted. For instance, as needs arise, one in which another layer is further laminated can be used.

C. Cell Culture Patterning Substrate

A cell culture patterning substrate according to the present invention will be explained. The cell culture patterning substrate according to the invention is one that has a base material and a cell culture pattern layer formed on the base material, wherein the cell culture pattern layer having a cell adhesion-inhibiting portion that at least contains a photocatalyst and a cell adhesion-inhibiting material that has cell adhesion-inhibiting properties that inhibit a cell from adhering and can be decomposed or modified owing to an action of a photocatalyst in combination with energy irradiation; and a cell adhesion portion that at least contains the photocatalyst and where the cell adhesion-inhibiting material is decomposed or modified.

The cell culture patterning substrate according to the present invention, as shown in, for instance, FIG. 3, has a base material 1 and a cell culture pattern layer 4 formed on the base material 1, and the cell culture pattern layer 4 has a cell adhesion portion 5 and a cell adhesion-inhibiting portion 6.

According to the present invention, since the cell culture pattern layer has the cell adhesion portion and the cell adhesion-inhibiting portion, the cell can be readily adhered only onto the cell adhesion portion, and even when cells are coated, for instance, over an entire surface of the cell culture pattern layer, only on the cell adhesion portion, the cell can be adhered in high precision.

In the present invention, as shown in, for instance, FIG. 4, in a pattern same as to the cell adhesion-inhibiting portion 6 of the cell culture pattern layer 4, a light-shielding portion 7 may be formed on the base material 1. When, by forming such light-shielding portion, the patterning substrate described, for instance, in the “B. Patterning substrate” is formed and when energy is irradiated from a base material side, the cell adhesion-inhibiting material only on a region where the light-shielding portion is not formed can be decomposed or modified; that is, the cell adhesion portion can be readily formed.

Hereinafter, the respective configurations of the cell culture patterning substrate according to the present invention will be explained.

1. Cell Culture Pattern Layer

A cell culture pattern layer used in the cell culture patterning substrate according to the present invention will be explained. The cell culture patter layer used in the invention is formed on the base material described below, and, as far as it contains a cell adhesion portion and a cell adhesion-inhibiting portion, is not particularly restricted. In particular, the cell culture pattern layer preferably contains a cell adhesive material or a binder. This is because when the cell adhesive material is contained, an adhesiveness with a cell of the cell adhesion portion can be made more excellent. Furthermore, this is because when the binder is contained, various characteristics such as the strength and durability can be imparted to the cell culture pattern layer.

The cell adhesion-inhibiting portion is a region that contains a photocatalyst and a cell adhesion-inhibiting material that has cell adhesion-inhibiting properties that inhibit a cell from adhering and can be decomposed and removed owing to an action of a photocatalyst in combination with energy irradiation; that is, a region where the adhesiveness with a target cell is low.

On the other hand, the cell adhesion portion is a region that at least contains the photocatalyst and where the cell adhesion-inhibiting material is decomposed or modified and the adhesiveness with a cell is high. Here, that the cell adhesion-inhibiting material is decomposed or modified means that the cell adhesion-inhibiting material is not contained or in comparison with an amount of the cell adhesion-inhibiting material contained in the cell adhesion-inhibiting portion the cell adhesion-inhibiting material is contained less. For instance, when the cell adhesion-inhibiting material is decomposed owing to an action of the photocatalyst in combination with energy irradiation, an amount of the cell adhesion-inhibiting material is not contained in the cell adhesion portion or a decomposition product of the cell adhesion-inhibiting material and the like is contained. Furthermore, when the cell adhesion-inhibiting material is modified owing to an action of the photocatalyst in combination with energy irradiation, a modified product and the like is contained in the cell adhesion portion.

In the cell adhesion portion, as mentioned above, the cell adhesive material having the adhesiveness with a cell is preferably contained. Thereby, the cell can be more excellently adhered onto the cell adhesion portion.

The cell adhesion-inhibiting material, photocatalyst, and binder are identical to those explained in the section of “A. Coating liquid for patterning substrate”; accordingly, explanations thereof are omitted. Hereinafter, a method of forming a cell culture pattern layer having the cell adhesion portion and the cell adhesion-inhibiting portion as mentioned above will be explained.

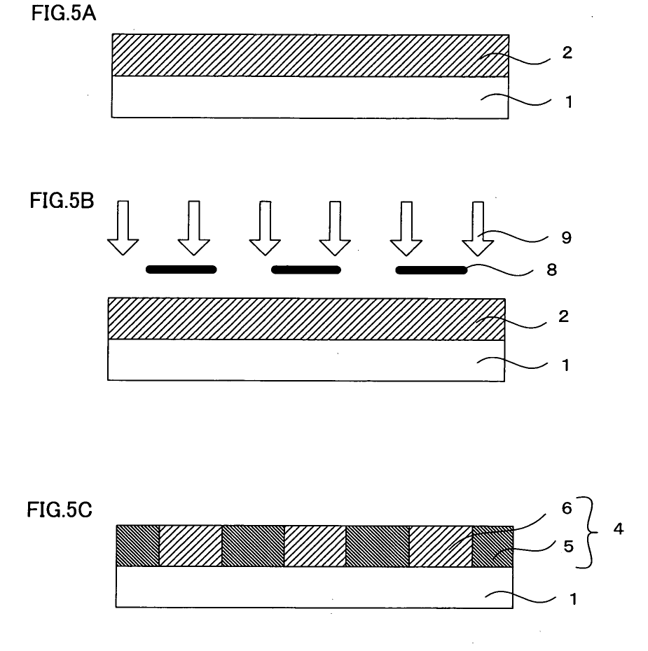

At the beginning, by using the coating liquid for patterning substrate explained in the “A. Coating liquid for patterning substrate” and the like, as shown in, for instance, FIGS. 5A to 5C, a cell culture patterning layer 2 that at least contains the photocatalyst and the cell adhesion-inhibiting material is formed on a base material 1 (FIG. 5A). Subsequently, energy 9 is irradiated on the cell culture patterning layer 2, in a pattern that forms the cell adhesion portion, by using, for instance, a photomask 8 and the like (FIG. 5B). Thereby, owing to an action of the photocatalyst contained in the cell culture patterning layer 2, the cell adhesion-inhibiting material in a region thereon energy is irradiated is decomposed or modified, and thereby a cell culture pattern layer 4 where the region where energy is irradiated becomes a cell adhesion portion 5 and a region where energy is not irradiated becomes a cell adhesion-inhibiting portion 6 is formed (FIG. 5C). The above-mentioned method of forming a cell culture patterning layer and the like are identical as the method explained in the “B. Patterning substrate”; accordingly, explanations thereof are not repeated. As mentioned above, when, together with the photocatalyst and the cell adhesion-inhibiting material, the cell adhesive material that is excellent in the adhesiveness with a cell is contained, the cell adhesion portion can be made a region that is more excellent in the adhesiveness with a cell.

The energy irradiation (exposure) mentioned in the present invention is a concept that includes all energy line irradiation that can decompose or modify the cell adhesion-inhibiting material owing to an action of the photocatalyst in combination with energy irradiation, and is not restricted to visible light irradiation.

Normally, a wavelength of light used in such energy irradiation is set in the range of 400 nm or less, and preferably in the range of 380 nm or less. This is because, as mentioned above, the photocatalyst that is preferably used as a photocatalyst is titanium dioxide, and as energy that activates a photocatalyst action by the titanium oxide, light having the above-mentioned wavelength is preferable.

As a light source that can be used in such energy irradiation, a mercury lamp, metal halide lamp, xenon lamp, excimer lamp and other various kinds of light sources can be cited.

Other than the method in which by use of the above-mentioned light source pattern irradiation is carried out through a photomask, a method of carrying out drawing irradiation in a pattern by use of laser such as excimer, YAG and the like and can be applied. Furthermore, when the base material has the light-shielding portion in a pattern same as that of the cell adhesion-inhibiting portion, energy can be irradiated over an entire surface from a base material side. In this case, there are advantages in that there are no needs of using the photomask and a process of positional alignment and the like.

An amount of irradiation of energy at the energy irradiation is an amount of irradiation necessary for decomposing or modifying the cell adhesion-inhibiting material owing to an action of the photocatalyst.

At this time, by irradiating energy with a layer that contains the photocatalyst heating, the sensitivity can be raised; accordingly, the cell adhesion-inhibiting material can be preferably and efficiently decomposed or modified. Specifically, it is preferable to heat in the range of 30 to 80° C.

The energy irradiation that is carried out through the photomask in the present invention, when the above-mentioned base material is transparent, may be carried out from either directions of a base material side or a side on which a cell culture pattern layer is formed. On the other hand, when the base material is opaque, it is necessary to apply energy irradiation from a side where the cell culture pattern layer is formed.

2. Base Material

A base material used for the cell culture patterning substrate according to the present invention will be explained. The base material used in the cell culture patterning substrate according to the invention, as far as it can form the cell culture pattern layer, is not particularly restricted. For instance, one on which the light-shielding portion is formed as mentioned above may be used.

As the base material, the light-shielding portion and the like that are used in the present invention, ones similar to those explained in the “B. Patterning substrate” can be used; accordingly, explanations thereof are not repeated here.

3. Cell Culture Patterning Substrate

A cell culture patterning substrate according to the present invention will be explained. The cell culture patterning substrate according to the invention, as far as the above-mentioned cell culture pattern layer is formed on the base material, is not particularly restricted. As needs arise, the cell culture patterning substrate may be the one as such that another layer may be formed thereon.

D. Cell Culture Substrate

Next, a cell culture substrate according to the present invention will be explained. The cell culture substrate according to the invention is one in which a cell are adhered onto the cell adhesion portion in the above-mentioned cell culture patterning substrate.

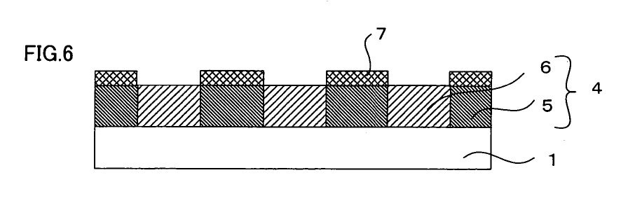

In the cell culture substrate according to the present invention, as shown in, for instance, FIG. 6, cells 7 are adhered only onto a cell adhesion portion 5 of a cell culture pattern layer 4, and, on a cell adhesion-inhibiting portion 6, cells 7 are not adhered.

According to the present invention, on the cell culture patterning substrate, a cell adhesion portion excellent in an adhesiveness with a cell and a cell adhesion-inhibiting portion that does not have the adhesiveness with a cell are formed. Accordingly, for instance, even when cells are coated over an entire surface of a cell culture patterning substrate, the cell can be adhered only to the cell adhesion portion and the cell on the cell adhesion-inhibiting portion can be readily removed. Thereby, without invoking a complicated process or using a processing liquid and the like that adversely affects on the cell, the cell culture substrate can be readily formed.

Hereinafter, the cell that are used in the cell culture substrate according to the present invention will be explained. Since an explanation of the cell culture patterning substrate is same as that in the “C. Cell culture patterning substrate”, here it is omitted.

(Cell)

As a cell used in the cell culture substrate according to the present invention, as far as it can adhere onto the cell adhesion portion of the cell culture patterning substrate but does not adhere to the cell adhesion-inhibiting portion, there is no particular restriction.

As the cell used in the present invention, except for, for instance, non-adhesive cells such as nervous tissue, liver, kidney, pancreas, blood vessel, brain, cartilage and blood corpuscle, all tissues present in an organism and cells derived therefrom can be used. Furthermore, since even for generally non-adhesive cells, recently, in order to adhere and fix, a technology of modifying a cell membrane is devised; accordingly, as needs arise, the non-adhesive cells, when this technology is applied, can be used in the invention.

The respective tissues such as mentioned above are formed of cells having various functions; accordingly, it is necessary to select a desired cell to use. For instance, in the case of the liver, it is formed of, other than hepatpcytes, epithelial cells, endothelial cells, Kupffer's cells, fibrocytes, and fat-storing cells and the like. In this case, since adhesion-inhibiting properties with a cell adhesion-inhibiting material is different depending on the kinds of the cells, in accordance with a cell strain, the cell adhesion-inhibiting material used in the cell adhesion-inhibiting portion and a composition ratio thereof have to be selected.

A method of adhering the cell to the cell adhesion portion, as far as it can adhere the cell only on the cell adhesion portion of the cell culture patterning substrate that has the cell adhesion portion and the cell adhesion-inhibiting portion, is not particularly restricted. For instance, the cell may be adhered by use of an ink jet printer, a manipulator or the like; however, a method in which after the cell suspension is disseminated to adhere the cell on the cell adhesion portion, unnecessary cells on the cell adhesion-inhibiting portion are washed with a phosphate buffer to remove the cells is generally used. As such a method, a method described in, for instance, a reference literature, Kevin E. Healy et al., “Spatial distribution of mammalian cells dicated by material surface chemistry”, Biotech. Bioeng. (1994), p. 792 can be used.

(Method of Manufacturing Cell Culture Substrate)

A method of manufacturing the cell culture substrate will be explained. The method of manufacturing the cell culture substrate in the present invention, as far as it can manufacture the cell culture substrate having a base material; a cell culture pattern layer that is formed on the base material and has the cell adhesion-inhibiting portion that at least contains a photocatalyst and a cell adhesion-inhibiting material that has cell adhesion-inhibiting properties that inhibit a cell from adhering and can be decomposed or modified owing to an action of the photocatalyst in combination with energy irradiation and a cell adhesion portion that at least contains the photocatalyst and where the cell adhesion-inhibiting material is decomposed or modified; and the cell adhered onto the cell adhesion portion, is not particularly restricted.

Such method of manufacturing the cell culture substrate may include such processes, for instance, coating a coating liquid for patterning substrate that contains the photocatalyst and the cell adhesion-inhibiting material on the base material to form the cell culture patterning layer; irradiating energy in a pattern on the cell culture patterning layer to form the cell culture pattern layer having a cell adhesion portion and a cell adhesion-inhibiting portion; and adhering the cell on the cell adhesion portion, and, as needs arise, other than the above processes, other processes may be included.

The photocatalyst, cell adhesion-inhibiting material, and base material and the like used in the method of manufacturing the cell culture substrate, and the method of forming the cell culture pattern layer and the like are same as to those explained in the “C. Cell culture patterning substrate”; accordingly, explanations thereof here are not repeated.

The present invention is not restricted to the above-mentioned embodiments. The above embodiments are only for exemplification, and all that have a configuration substantially same as a technical idea described in the range of claims of the invention and exhibit identical operations and effects are included in a technical range of the invention.

### EXAMPLES

Hereinafter, examples and comparative examples are shown and thereby the present invention will be more specifically described.

### Example 1

(Preparation of Cell Culture Patterning Substrate)

Blended were 3 g of isopropyl alcohol, 0.4 g of organosilane (trade name: TSL8114, manufactured by GE Toshiba Silicones), 0.04 g of fluoroalkylsilane (trade name: TSL8233, manufactured by GE Toshiba Silicones), and 1.5 g of photocatalytic inorganic coating agent (trade name; ST-K01, manufactured by ISHIHARA SANGYO KAISHA, LTD.), followed by heating at 100° C. for 20 min while stirring.

This solution was coated by a spin coating method on a quartz glass substrate processed alkali treatment beforehand, the substrate was heated at 150° C. for 10 min to dry, followed by forwarding a hydrolysis and a polycondensation reaction, and thereby a patterning substrate having a cell culture patterning layer in which a photocatalyst is solidly fixed in organo-polysiloxane and that has a film thickness of 0.2 μm was obtained.

On the substrate, by use of a photomask, UV exposure was carried out for 900 sec at an illuminance of 300 mW/cm² with a mercury lamp (wavelength: 365 nm), and thereby a cell culture patterning substrate having the pattern in which a non-exposed portion is a cell adhesion-inhibiting portion and an exposed portion is a cell adhesion portion was obtained.

(Adhering Cells)

A procedure and the like of the cell culture was, based on a method described in, for instance, Asakura Shoten, “Soshikibaiyo no Gijyutsu, Dai San Han, Kiso”, the Japanese Tissue Culture Association(editor), carried out as follows.

A liver removed from a rat was transferred in a petri dish and chopped into 5 mm size with a surgical knife, followed by adding 20 ml of DMEM culture media and lightly suspending with a pipette, further followed by filtrating with a cell filter. An obtained coarse cell dispersed suspension liquid was centrifuged at 500 to 600 rpm for 90 s, followed by suctioning a supernatant to remove. To remaining cells, a DMEM culture media was added anew, followed by centrifuging again. This operation was repeated three times, and thereby substantially homogeneous hepatpcytes were obtained. To the obtained hepatpcytes, 20 ml of the DMEM culture media was added to suspend, and thereby a hepatpcyte suspension was prepared.

To 14.12 g of Waymouth MB752/1 culture media (containing L-glutamine and lacking NaHCO₃) (manufactured by GIBCO), 900 ml of distilled water was added. Thereto, 2.24 g of NaHCO₃, 10 ml of amphotericin B (ICN) and 10 ml of penicillin streptomycin liquid (manufactured by GIBCO) were added, followed by agitating. After this solution was controlled to pH 7.4, a total amount was adjusted to 1000 ml, followed by filtrating and sterilizing with a membrane filter of 0.22 μm, and thereby a Waymouth MB752/1 culture media liquid was prepared.

The previously prepared hepatpcyte suspension was suspended in the previously prepared Waymouth MB752/1 culture media liquid, followed by disseminating on the above-mentioned cell culture patterning substrate having a cell adhesion portion and a cell adhesion-inhibiting portion. The substrate was stood still in a 5% CO₂-gassed incubator at 37° C. for 24 hr to adhere hepatpcytes over an entire surface of the substrate. The substrate was washed twice with PBS to remove non-adhesion cells and died cells, followed by replacing by a new culture media liquid.

While replacing the culture media liquid, the cells were cultured on up to 48 hr, followed by observing cells by an optical microscope, and it was confirmed that cells adhered on the cell culture patterning substrate along the cell adhesion portion.

### Example 2

Except that in place of a quartz substrate used in example 1, a quartz substrate provided thereon with a striped light-shielding layer having a light-shielding portion of 80 μm and a space portion of 300 μm was used and, without using a photomask and the like, UV light was irradiated from a back surface side of the substrate, similarly to example 1, a cell culture patterning substrate was prepared and cells were cultured.

In this case too, when the cells were observed under an optical microscope, it was confirmed that cells adhered on the cell culture patterning substrate along a cell adhesion portion.

### Example 3

(Preparation of Cell Culture Patterning Substrate)

Blended were 3 g of isopropyl alcohol, 0.4 g of organosilane (trade name: TSL8114, manufactured by GE Toshiba Silicones), 0.1 g of MPC polymer (trade name: Lipidure-PMB, manufactured by Nippon Oil & Fats Corporation), and 1.5 g of photocatalytic inorganic coating agent (trade name; ST-K01, manufactured by ISHIHARA SANGYO KAISYA, LTD.), followed by heating at 100° C. for 20 min while stirring.

This solution was applied by means of a spin coating method on a previously alkali-treated quartz glass substrate, the substrate was heated at a temperature of 150° C. for 10 min to dry, followed by conducting a hydrolysis and a polycondensation reaction, and thereby a patterning substrate having a cell culture patterning layer in which a photocatalyst was solidly fixed in organo-polysiloxane and that has a film thickness of 0.17 μm was obtained.

Preparing a cell culture patterning substrate and adhering cells were carried out similarly to example 1. As a result thereof, it was confirmed that cells adhered on the cell culture patterning substrate along a portion where MPC polymer was exposed.

1. A patterning substrate, comprising:
a base material; and
a cell culture patterning layer that is formed on the base material and at least contains a photocatalyst and a cell adhesion-inhibiting material that has cell adhesion-inhibiting properties that inhibit a cell from adhering and is decomposed or modified owing to an action of the photocatalyst in combination with energy irradiation.

2. The patterning substrate according to claim 1, wherein a light-shielding portion is formed in a pattern on the base material.

3. The patterning substrate according to claim 1, wherein the cell culture patterning layer contains a binder.

4. The patterning substrate according to claim 2, wherein the cell culture patterning layer contains a binder.

5. The patterning substrate according to claim 1, wherein the cell culture patterning layer contains a cell adhesive material having an adhesiveness with a cell at least after energy is irradiated.

6. The patterning substrate according to claim 2, wherein the cell culture patterning layer contains the cell adhesive material having the adhesiveness with a cell at least after energy is irradiated.

7. The patterning substrate according to claim 3, wherein the cell culture patterning layer contains the cell adhesive material having the adhesiveness with a cell at least after energy is irradiated.

8. The patterning substrate according to claim 4, wherein the cell culture patterning layer contains the cell adhesive material having the adhesiveness with a cell at least after energy is irradiated.

9. A cell culture patterning substrate comprising:
a base material; and
a cell culture pattern layer formed on the base material,
wherein the cell culture pattern layer contains a cell adhesion-inhibiting portion that at least contains a photocatalyst and a cell adhesion-inhibiting material that has cell adhesion-inhibiting properties that inhibit a cell from adhering and is decomposed or modified by an action of the photocatalyst in combination with energy irradiation; and a cell adhesion portion that at least contains the photocatalyst and where the cell adhesion-inhibiting material is decomposed or modified.

10. The cell culture patterning substrate according to claim 9, wherein a light-shielding portion is formed on the base material in a pattern same as that of the cell adhesion-inhibiting portion.

11. The cell culture patterning substrate according to claim 9, wherein the cell culture pattern layer contains a binder.

12. The cell culture patterning substrate according to claim 10, wherein the cell culture pattern layer contains a binder.

13. The cell culture patterning substrate according to claim 9, wherein the cell culture pattern layer contains a cell adhesive material having an adhesiveness with a cell at least after energy is irradiated.

14. The cell culture patterning substrate according to claim 10, wherein the cell culture pattern layer contains the cell adhesive material having the adhesiveness with a cell at least after energy is irradiated.

15. The cell culture patterning substrate according to claim 11, wherein the cell culture pattern layer contains the cell adhesive material having the adhesiveness with a cell at least after energy is irradiated.

16. The cell culture patterning substrate according to claim 12, wherein the cell culture pattern layer contains the cell adhesive material having the adhesiveness with a cell at least after energy is irradiated.

17. A cell culture substrate, wherein the cell is adhered onto the cell adhesion portion in the cell culture patterning substrate according to claim 9.

18. A coating liquid for patterning substrate at least comprising:
a photocatalyst; and
a cell adhesion-inhibiting material that has cell adhesion-inhibiting properties that inhibit a cell from adhering and is decomposed or modified owing to an action of the photocatalyst in combination with energy irradiation.

19. The coating liquid for patterning substrate according to claim 18, wherein the coating liquid contains a binder.

20. The coating liquid for patterning substrate according to claim 18, wherein the coating liquid contains a cell adhesive material having an adhesiveness with a cell at least after energy is irradiated.
